(19) United States
(12) Patent Application Publication
(10) Pub. No.: US 2016/0003802 A1
(43) Pub. Date: Jan. 7, 2016

(54) Module for Electronic Musical Instrument, and Electronic Musical Instrument

(71) Applicants: Shota SAITO, Ome-shi Tokyo (JP); Yuji FUKAISHI, (US)

(72) Inventors: Shota Saito, Ome-shi Tokyo (JP); Yuji Fukaishi, Atsugi-shi Kanagawa (JP)

(21) Appl. No.: 14/768,980

(22) PCT Filed: Feb. 20, 2014
(86) PCT No.: PCT/JP2014/054088

§ 371 Date: Aug. 19, 2015

(30) Foreign Application Priority Data
Feb. 21, 2013 (JP) 2013-032446

(51) Int. Cl. G01N 33/497; G10H 1/053

(52) U.S. Cl. CPC G01N 33/4972; G10H 1/053

(57) ABSTRACT

The invention provides a module for electronic musical instruments and an electronic musical instrument that is easy to use during drinking and allow a user to know the alcohol concentration in their exhaled breath in a timely manner. The electronic musical instrument of the present invention can easily be used by a user while drinking. Accordingly, the user and the audience can know the alcohol concentration in the user's exhaled breath in timely manner.

### FIELD OF THE INVENTION

The present invention relates to an electronic musical instrument modulating a waveform of a sound based on the alcohol concentration of a user and a module for that.

### BACKGROUND OF THE INVENTION

Generally, a banquet is often held in a restaurant providing alcohols such as bars or a house by a large number of people and also accompanied by drinking alcohol at the same time. Drinking excessive quantities of alcohol is not only bad for people's own health, but also bothers people who do not drink alcohol by a bad breath and furthermore they may cause trouble to other people by hassling them. It should also be understood that driving a car is forbidden.

However, it is difficult to control the quantity of drinking by taking the same quantity of the drinking with another person without knowing their own drinking quantity, because people drinking in a banquet are usually deep in a conversation with another person. If their own quantity of drinking can be known during drinking, they are able to control the quantity of the drinking adjusting to themselves because it is capable to limit the drinking quantity to prevent excessive drinking when they drink too much, and drink without anxiety when they drink less adversely.

In the meantime, portable instruments for measuring alcohol concentration are sold currently. There is a measuring instrument on the market that is smaller than a cell-phone and needs less power consumption, and it is also easy to operate and allows to measure only by blowing a breath lightly and its measurement speed is less than ten seconds. The background that enables such high-performance and downsizing depends, to a large extent, on the utilization of the improved semiconductor gas sensor.

For example, as an application relating to the instrument for measuring a gas concentration generated from the human body using the semiconductor gas sensor, the patent document 1 discloses techniques about instrument for measuring a bad smell that measures a presence of a bad breath and a concentration of bad smell materials included in an exhaled breath generated from a human body using the semiconductor gas sensor. This instrument for measuring a bad smell measures and displays exhaled bad breath materials, but does not applied to others by using a measured result.

In addition, the patent document 2 discloses techniques in relation to an instrument for measuring an alcohol concentration which has a sensor for detecting an alcohol concentration in an exhaled breath using the semiconductor gas sensor. The instrument for measuring an alcohol concentration is used to measure a degree of the influence of the alcohol and does not apply to others by using a measured result.

Moreover, tools generally called party goods are brought and often warm up in the drinking spot such as the banquet. However, there are few party goods combined with drinking itself though they are used mainly in drinking spot.

It is not party goods, but, for example, the patent document 3 discloses a karaoke microphone with a function of an alcohol concentration measurement and a karaoke system using it. This system makes a karaoke microphone accompanied by an alcohol concentration measurement means, and displays the measurement data of the measured alcohol concentration thereof on a display device with a karaoke performance device in a scheduled timing while a user sings songs using this karaoke microphone.

However, the system described in the patent document 3 shows only the alcohol concentration to the user and do not make use of it to others by using the alcohol concentration of the user.

At the banquet, sometimes, musical instruments are also used to present a performance and play or accompany songs such as karaoke. Such musical instruments are not combined with either the quantity of drinking or the behavior of drinking.

Now, an example of conventional electronic musical instruments is described with reference to FIG. 1. FIG. 1 is a block diagram of a synthesizer 100 which is an example of conventional electronic musical instruments.

The synthesizer 100 is comprised of a control circuit board 101, a keyboard 102 and a speaker 103. The control circuit board 101 is comprised of a detection unit 111 comprising an interval detection unit 121, a harmony detection unit 122, a tempo detection unit 123 and a volume detection unit 124, a waveform data generation unit 112, an amplification unit 113, a CPU 114, a ROM 115 and a RAM 116. Each part on the control circuit board 101 is connected to a BUS.

The CPU 114 controls the whole control circuit board 101 by performing a control program stored in the ROM 115. In the ROM 115, a program to operate the CPU 114, various data necessary to generate waveform data of a sound and others are stored. In the RAM 116, various programs and data necessary for control by the CPU 114 are stored temporarily.

When a key of keyboard 102 is pushed, from the position of a pushed key, an interval detection unit 121 detects an interval and a harmony detection unit 122 detects whether it is a harmony or not and a combination of sounds if it is a chord, and transmit the result of the detection to the waveform data generation unit 112. Furthermore, the tempo detection unit 123 detects a tempo (pace) of the sound from a length and a timing of the key being pushed, and transmit the result of the detection to the waveform data generation unit 112. The tempo of the sound comprises, for example, the length of the sound and the distance between the previous and next sounds. The volume detection unit 124 detects the volume from strength of the key being pushed, and transmit the result of the detection to the waveform data generation unit 112.

The waveform data generation unit 112 make the waveform data of the sound based on the data received from each part of the detection unit 111 and transmits it to the amplification unit 113. The amplification unit 113 amplifies it based on the received waveform data and outputs it to the speaker 103.

Thus, the conventional electronic musical instruments output the sound that reflected faithfully the interval, the chord, the tempo and the volume of the sound input by the keyboards from the speaker 103.

### PRIOR ART DOCUMENTS

### Patent Document

Patent document 1 Japanese published unexamined application No. Sho 64-35368

Patent document 2 Japanese published examined application No. Hei 07-33162

Patent document 3 Japanese published unexamined application No. 2005-242062

### SUMMARY OF THE INVENTION

### Problem to be Solved by the Invention

The conventional instrument for measuring bad smell, the instrument for measuring the alcohol concentration and the karaoke microphone which have the function for measuring the alcohol concentration as described above measure the concentration of a bad smell of materials or the alcohol concentration in a exhaled breath and only display a result thereof to a user, and their entertainment property is very low. The conventional instrument for measuring alcohol concentration requires people to stop a conversation with another person during a banquet many times, though the alcohol concentration in people's own exhaled breaths must be measured during drinking many times to regulate their alcohol intake. Therefore, they cannot know the alcohol concentration in their exhaled breaths in an appropriate timing, and it is difficult to suitably regulate their alcohol intake.

Even if the conventional instrument for measuring an alcohol concentration is high-performance and downsized, it is required only when people drink, but in usual times because it includes only a function to measure the alcohol concentration. However, the drinking may not be necessarily planned and may be suddenly needed. Therefore, there is an inconvenience that the instrument for measuring the alcohol concentration must be always carried, even though it is needed only at the time of drinking.

Additionally, because the instrument for measuring the alcohol concentration is not became widespread generally, there is extremely little number of people to possess it, and there is a situation that most of people who drink do not recognize the quantity of their alcohol intake as the quantitative numerical value.

Additionally, the function to measure the alcohol concentration is not included in the conventional musical instrument used during a banquet, and a user cannot know their own quantity of drinking.

Thus the present invention was made in view of the problem that the conventional art has, and it is the purpose of the present invention to provide a module for electronic musical instruments and an electronic musical instrument wherein a user can use easily and know the alcohol concentration in their exhaled breath in an appropriate timing during drinking.

### Means to Solve the Problem

In view of the problem described above, the inventors found the idea to make an electronic musical instrument accompanied by an alcohol concentration measurement means, and measure an alcohol concentration in a user's exhaled breath while the user uses the electronic musical instrument, and change a waveform of a sound appearing from the electronic musical instrument in real time according to the alcohol concentration by using the measurement data thereof. The electronic musical instrument wherein the sound changes depending on the alcohol concentration is unprecedented and their entertainment property is high, and then the inventors think that the problem could be solved which can be easily used by the user during drinking and know the alcohol concentration in their exhaled breath in an appropriate timing, and the present invention is come to be created.

That is to say, the present invention provides a module for electronic musical instruments comprising a an alcohol concentration detection means for detecting an alcohol concentration in an exhaled breath of a user and a modulation means for modulating a waveform of a sound based on the alcohol concentration detected by the means for detecting the alcohol concentration as described above.

The present invention also provides an electronic musical instrument comprising an input means for inputting the sound to be output, an alcohol concentration detection means for detecting an alcohol concentration in an exhaled breath of a user and a modulation means for modulating a waveform of a sound to be output which is input to the means for inputting described above based on the alcohol concentration detected by the means for detecting the alcohol concentration.

The present invention also provides the module for electronic musical instruments or the electronic musical instrument, wherein the modulating means described above modulates the waveform of the sound by changing at least one of those selected from the group consisting of an interval, a chord, a tempo, a volume and a kind of the musical instrument in the module for electronic musical instruments or the electronic musical instrument as described above.

The present invention also provides the module for electronic musical instruments or the electronic musical instrument as described above, further comprising means for controlling an external air conditioning system based on the alcohol concentration detected by the means for detecting the alcohol concentration described above.

The present invention also provides the module for electronic musical instruments or the electronic musical instrument as described above, further comprising light emitting means included in the electronic musical instrument or external light emitting means, or light control means for controlling both lights based on the alcohol concentration detected by the means for detecting the alcohol concentration as described above.

### Effect of the Invention

The present invention is an electronic musical instrument comprising means for detecting the alcohol concentration and means for modulating a waveform, and modulates a waveform of a sound based on an alcohol concentration in an exhaled breath of a user. The electronic musical instrument of the present invention has very high entertainment property since a sound changes depending on the quantity of drinking of the user, and is easy to use during a banquet. Additionally, the user and the audience can know easily the quantity of drinking of the user by simply hearing the sound of the electronic musical instrument. Thus, the present invention can effectively measure the quantity of drinking of the user (the alcohol concentration in the exhaled breath) without giving the user any load. Therefore, the user can use it easily during drinking and know the alcohol concentration in their exhaled breath in an appropriate timing and suitably adjust the alcohol intake.

### BRIEF DESCRIPTION OF THE DRAWINGS

FIG. 1 is a block diagram of a synthesizer 100 which is an example of the conventional electronic musical instrument.

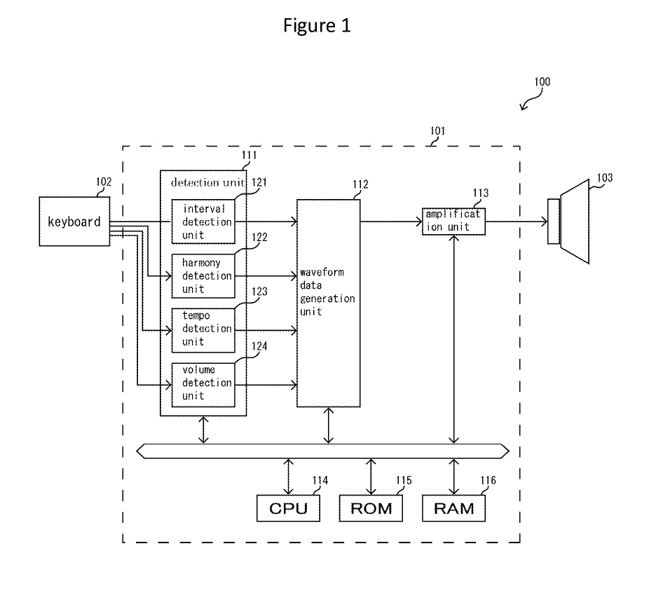

FIG. 2 is a block diagram of an electrical flute 1 which is one exemplary embodiment of the electronic musical instrument of the present invention.

FIG. 3 is a figure representing the one embodiment of the electronic musical instrument of the present invention.

FIG. 4 is a flow chart which shows an example of a flow of a process when the electrical flute of the present invention is used.

FIG. 5 is a block diagram of an electrical flute 60 which is another embodiment of the electronic musical instrument of the present invention.

FIG. 6 is a flow chart which shows an example of a flow of a process when the electrical flute of the present invention is used.

FIG. 7 is a block diagram of an electrical flute 70 which is another embodiment of the electronic musical instrument of the present invention.

FIG. 8 is a flow chart which shows an example of a flow of a process when the electrical flute of the present invention is used.

### MODE FOR CARRYING OUT THE INVENTION

### First Embodiment

### Structure of the Electrical Flute 1 and the Electrical Flute Module 2

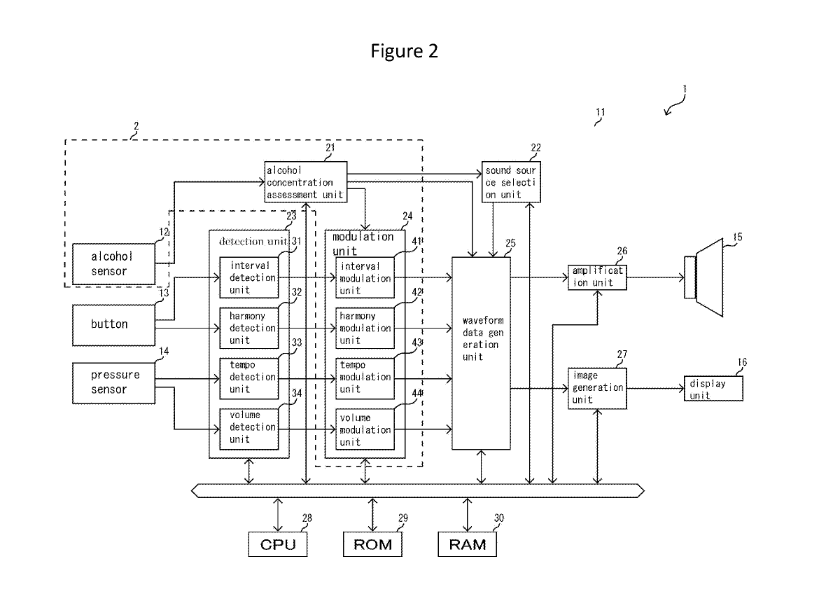

A module for electronic musical instruments and an electronic musical instrument of the present invention are described in detail with one embodiment as an example as follows. FIG. 2 is a block diagram of an electrical flute 1 which is one embodiment of the electronic musical instrument of the present invention.

The electrical flute 1 (electronic musical instrument) is comprised of a control circuit board 11, an alcohol sensor 12 (means for detecting the alcohol concentration), a button 13 (input means), a pressure sensor 14 (input means), a speaker 15 (output means) and a display unit 16. The control circuit board 11 is comprised of an alcohol concentration assessment unit 21, a sound source selection unit 22, a detection unit 23, a modulation unit 24 (modulating means), a waveform data generation unit 25, an amplification unit 26, an image generation unit 27 and a CPU 28, a ROM 29 and a RAM 30. The detection unit 23 comprises an interval detection unit 31, a harmony detection unit 32, a tempo detection unit 33 and a volume detection unit 34. The modulation unit 24 comprises an interval modulation unit 41, a harmony modulation unit 42, a tempo modulation unit 43 and a volume modulation unit 44. Each part on the control circuit board 11 is connected to a BUS.

The electrical flute module 2 (module for electronic musical instruments) is comprised of the alcohol sensor 12, the alcohol concentration assessment unit 21 and the modulation unit 24 in the components of the electrical flute 1. The electrical flute module 2 is a module giving an electrical flute a function to measure an alcohol concentration and a function to modulate a sound output from an electrical flute based on an alcohol concentration of a user.

The CPU 28 controls the whole control circuit board 11 by performing control programs stored in the ROM 29. In the ROM 29, programs to operate the CPU 28 and various data necessary to make the waveform data of the sound are stored. In the RAM 30, various programs necessary for control by the CPU 28 and data are stored temporarily.

The button 13 is a switch to indicate the output of the sound of the peculiar interval. More than one button 13 may be provided and each of them may respond to the sound of the different interval. The pressure sensor 14 is a sensor detecting a pressure value of an exhaled breath blown from a mouthpiece which is not shown. The button 13 and the pressure sensor 14 are the input means that are inputted a sound desired to output (a sound to be output) from the electrical flute 1 by a user. The user can input an interval peculiar to the button 13 as an interval of the sounds to be output by blowing the exhaled breath from the mouthpiece which is not shown while pushing the button 13. Furthermore, by the strength and the length of the blowing exhaled breath, the sound strength and length can be input. Note that the button 13 may be comprised to output the sound of the different intervals, for example a halftone, between the positions where it is pushed halfway and completely.

Each part of the detection unit 23 detects an interval, a chord, a tempo and a volume of the sound to be output which a user input through the button 13 and the pressure sensor 14.

Specifically, when the exhaled breath was blown from the mouthpiece of the electrical flute 1 which is not shown, from a pushed position of the button 13 and a combination thereof, the interval detection unit 31 detects an interval and the harmony detection unit 32 detects whether it is a harmony and a combination of sounds if it is a chord. The tempo detection unit 33 detects a tempo (pace) of a sound from a length of time and distance which the pressure sensor 14 detects a pressure value. The tempo of the sound comprises, for example the length of the sound, the distance between the previous and next sounds. The volume detection unit 34 detects a volume from the pressure value that the pressure sensor 14 detected.

The electrical flute 1 may comprise a pressure value assessing means which is not shown, between the pressure sensor 14 and the detection unit 23. The pressure value assessing means judges whether the pressure value detected by the pressure sensor 14 is bigger than a sounding threshold, and transmits the pressure value to the detection unit 23 when it is bigger and does not transmit when it is smaller than or equal to the sounding threshold. The sounding threshold is a threshold to judge whether a blown exhaled breath has enough pressure to output a sound. Thereby, the sound is output only when the pressure value of the blown exhaled breath is bigger than the sounding threshold. The sounding threshold may be stored in a memory unit which is not shown.

The interval detection unit 31, the harmony detection unit 32, the tempo detection unit 33 and the volume detection unit 34 convert the detected interval, chord, tempo and volume into the data to generate a waveform of a sound and transmit it to the interval modulation unit 41, the harmony modulation unit 42, the tempo modulation unit 43 and the volume modulation unit 44, respectively. For example, the interval and the harmony are converted into the data indicating a frequency of the sound, and the tempo is converted into the data indicating the timing of the appearance and the disappointment of the waveform, and the volume is converted into the data indicating the amplitude of the waveform.

The alcohol sensor 12 is a sensor detecting an alcohol concentration in an exhaled breath of a user blown from a mouthpiece and transmits detected alcohol concentration to the alcohol concentration assessment unit 21. For example, a known semiconductor gas sensor can be used for the alcohol sensor 12.

The alcohol concentration assessment unit 21 generates a modulating signal to modulate a waveform of a sound based on the alcohol concentration detected by the alcohol sensor 12 and transmits it to the modulation unit 24. When the modulating signal is received, the modulation unit 24 modulates the waveform of the sound detected by the detection unit 23. Thereby, the modulation unit 24 can modulate the waveform of the sound based on the alcohol concentration.

For example, when an alcohol concentration is higher than a predetermined modulation threshold, the alcohol concentration assessment unit 21 may transmit a modulating signal to the modulation unit 24. In this case, the waveform of the sound is modulated when the alcohol concentration are higher than the modulation threshold, and the input sound is output with no change because the modulating signal is not transmitted when it is smaller than or equal to the modulation threshold. Therefore, a user can know that their own quantity of drinking exceeds a predetermined quantity by hearing the modulated sound.

The modulation threshold may be stored in the memory unit which is not shown and may be input by the input means not shown.

More than one modulation threshold may also be set, for example it may be set in a stepwise fashion. In this case, the alcohol concentration assessment unit 21 may transmit the different modulating signal depending on that the alcohol concentration is between any two modulation thresholds of a plurality of modulation threshold. The modulating signal may be a signal to modulate the waveform of the sound by a peculiar modulation method, respectively. The modulation unit 24 modulates the waveform of the sound using the modulation method corresponding to the received modulating signal. The modulation method corresponding to the modulating signal may be stored in the memory unit which is not shown.

The output sound may also be modulated based on the alcohol concentration continually and may be modulated at random, and not limited to the embodiment described above.

“Modulating a waveform” herein means to change a shape of a waveform. For example, a waveform is modulated by changing an amplitude, frequency and phase of the waveform, and changing the timing of the appearance and the disappearance of the waveform, and adding another waveform or replacing with another waveform.

Specifically, the modulation unit 24 can modulate a waveform of the sound by converting the data received from the detection unit 23 into another data according to a program stored previously in the memory unit which is not shown, when a modulating signal is received.

More specifically, for example, the interval modulation unit 41 and the harmony modulation unit 42 changes the frequency of the sound indicated by the received data according to a program, and make the new data indicating the combination of different interval and chords. Moreover, for example, the tempo modulation unit 43 changes the timing of the appearance and the disappearance of the waveform indicated by the received data according to the program, and make the new data indicating the length and the timing of the different sound. Furthermore, for example, the volume modulation unit 44 changes an amplitude indicated by the received data according to the program, and make the new data indicating the different volume. Then, the new data are transmitted to the waveform data generation unit 25 after these conversions.

Note that, when the modulation unit 24 receives the modulating signal, at least one of the interval modulation unit 41, the harmony modulation unit 42, the tempo modulation unit 43 and the volume modulation unit 44 should convert the data. Therefore, for example, only the interval may be modulated, and only the interval and the volume may be modulated.

When the modulating signal is not received, each part of the modulation unit 24 just transmits the data received from each part of the detection unit 23 to the waveform data generation unit 25 without converting.

The sound source selection unit 22 selects a kind of musical instruments which is a sound source and makes a signal indicating the kind of the selected musical instrument to transmit it to the waveform data generation unit 25. The kind of the selected musical instrument may be set previously and may be input by the input means which is not shown.

When the alcohol concentration is higher than the modulation threshold, the alcohol concentration assessment unit 21 may transmit the modulating signal to the sound source selection unit 22, but not the modulation unit 24. In this case, the sound source selection unit 22 becomes the modulating means. When the modulating signal is received, the sound source selection unit 22 selects the sound source different from the sound source that should be selected. For example, in case the modulating signal is received when the piano is input as the sound source to be selected from the input means which is not shown, it may be comprised to select a violin.

When the alcohol concentration are higher than the modulation threshold, the alcohol concentration assessment unit 21 may also transmit the modulating signal to both the modulation unit 24 and the sound source selection unit 22.

That is to say, the modulating means of the present invention may modulate the waveform of the sound by changing at least one selected from a group consisting of the interval, the chord, the tempo, the volume and the kinds of musical instruments.

The waveform data generation unit 25 makes the waveform data of the sound using the selected sound source based on the data received from each part of the modulation unit 24 and the sound source selection unit 22, and transmits it to the amplification unit 26. The amplification unit 26 amplifies it based on the received waveform data to output it to the speaker 15. The speaker 15 outputs the amplified sound.

Note that, when the alcohol concentration is smaller than or equal to the modulation threshold, the alcohol concentration assessment unit 21 may transmit a silent signal to the waveform data generation unit 25 in order not to output the sound. When the silent signal is received, the waveform data generation unit 25 does not transmit any waveform data to the amplification unit 26. Thereby, the electrical flute outputting the sound only when a user drinks to some extent can be provided.

The alcohol concentration assessment unit 21 may also transmit an alarm signal to the waveform data generation unit 25 when an alcohol concentration is higher than a predetermined warning threshold and transmit a modulating signal to the modulation unit 24 when the alcohol concentration is smaller than or equal to the warning threshold and is higher than the modulation threshold. The warning threshold is a threshold to determine whether a quantity of drinking is excessive, and is set in a value higher than the modulation threshold.

The warning threshold may be stored in the memory unit which is not shown, and may be the input by a user using the input means which is not shown.

When the alarm signal is received, the waveform data generation unit 25 may transmit the waveform data of the alarm sound to warn the user that the quantity of drinking is excessive to the amplification unit 26. The waveform data of the alarm sound may be stored previously in the memory unit which is not shown. Thereby, the alarm sound is output in place of the sound which is generated based on the data from the modulation unit 24.

When the alarm signal is received, the waveform data generation unit 25 may also transmit an image generating signal to produce a warning image warning a user that the quantity of drinking is excessive to the image generation unit 27. The image generation unit 27 outputs the warning image to the display unit 16 based on the image generating signal. The warning image may be stored previously in the memory unit which is not shown. The warning image should be the indication that can be confirmed visually and may be, for example, an image displaying a message or a picture promoting to suppress the quantity of drinking.

When the alarm signal is received, the waveform data generation unit 25 may also transmit the image generating signal to the image generation unit 27 while it transmits the waveform data of the alarm sound to the amplification unit 26.

The waveform data generation unit 25 may also control an outside air conditioning system. For example, the waveform data generation unit 25 may switch on or off the air conditioner of the room corresponding to the alcohol concentration of the user based on the signal from the alcohol concentration assessment unit 21 or change the preset temperature.

The electrical flute 1 may also comprise a warning light which is not shown, and may make this warning light on when the waveform data generation unit 25 receives the alarm signal. For example, LED can be used for a warning light.

The electrical flute 1 may also comprise an alcohol mode switch which is not shown. The alcohol mode switch is a switch to change a normal mode and an alcohol mode. The normal mode is the mode that a sound corresponding to the pushed button 13 is output with no change regardless of the alcohol concentration, when the electrical flute 1 is played. The alcohol mode is the mode that the waveform of the sound is modulated depending on the alcohol concentration in the exhaled breath as described above.

The alcohol mode switch may be provided on a signal line connecting, for example the alcohol sensor 12 and the alcohol concentration assessment unit 21. That is to say, the signal line connecting the alcohol sensor 12 and the alcohol concentration assessment unit 21 may be off on the normal mode, and the alcohol sensor 12 and the alcohol concentration assessment unit 21 may be connected when the alcohol mode switch is on.

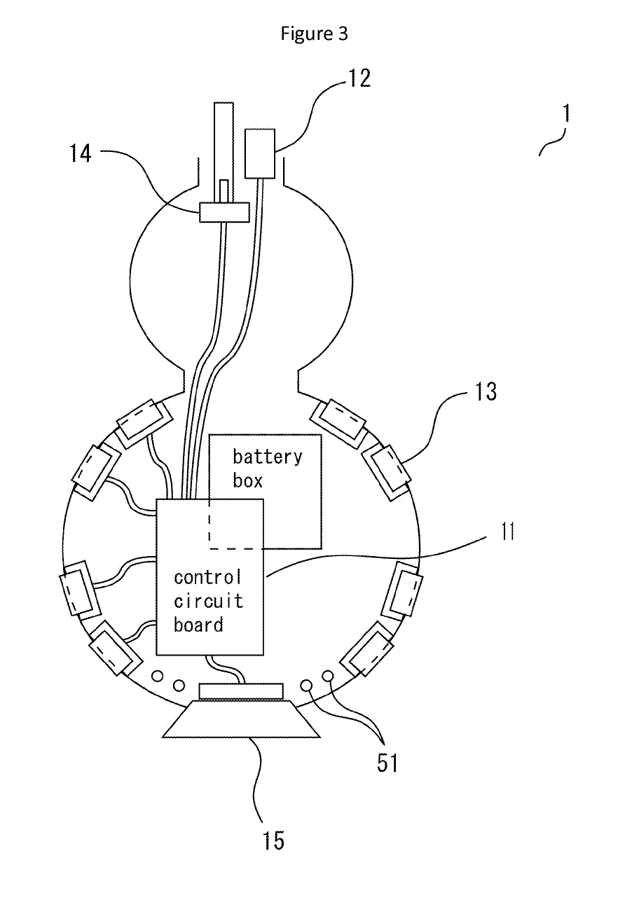

Then, one embodiment of the electrical flute of the present invention is described. FIG. 3 is a figure representing one embodiment of the electronic musical instrument of the present invention. The electrical flute 1 shown in FIG. 3 has a shape of a gourd which is connected two spheres, and one sphere is provided with a mouthpiece, and the other sphere is provided with a plurality of buttons 13, a speaker 15 and warning lights 51. The electrical flute 1 comprises an alcohol sensor 12 and a pressure sensor 14 near the mouthpiece. The inside of the electrical flute 1 is hollow, and a control circuit board 11 and a battery box are installed.

Specific Example of the Flow of the Process

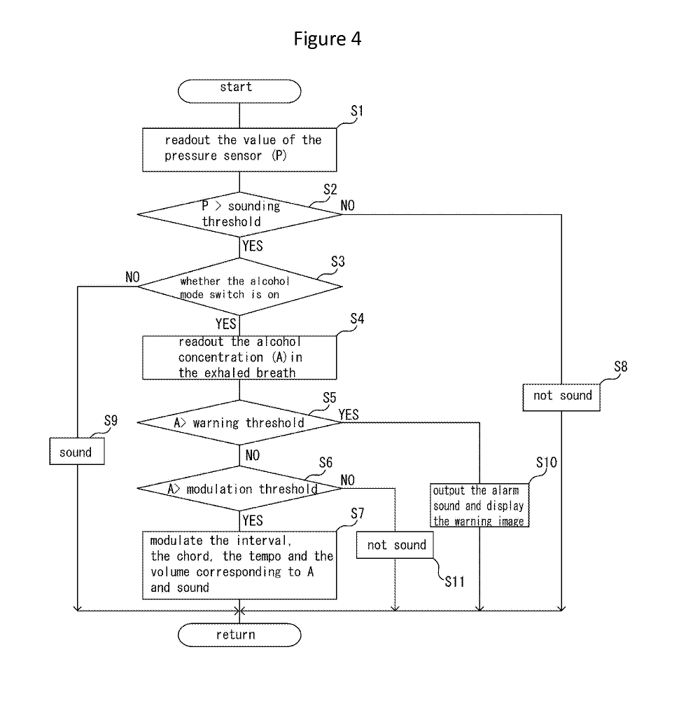

The specific example of the flow of the process from playing the electrical flute 1 to outputting the sound is described. FIG. 4 is a flow chart showing an example of the flow of the process when the electrical flute of the present invention is used. Note that it is described here when a pressure value assessing means is included between the pressure sensor 14 and the detection unit 23, and an alcohol mode switch is included between the alcohol sensor 12 and the alcohol concentration assessment unit 21.

When an exhaled breath is blown into the mouthpiece of the electrical flute 1 by a user, the pressure sensor 14 detects the pressure value (P) of the blown exhaled breath to transmit it to the pressure value assessing means which is not shown (step S1).

Then the pressure value assessing means assessed whether P is bigger than a sounding threshold (step S2). When P is smaller than or equal to the sounding threshold (NO), the pressure value is not transmitted to the detection unit 23, and there is no sounding from the electrical flute 1 (step S8), and the process is finished. Meanwhile, when P is bigger than the sounding threshold (YES), the process moves to step S3.

In step S3, the alcohol concentration assessment unit 21 assesses whether the alcohol mode switch is on (step S3). When it is not on (NO), the alcohol concentration assessment unit 21 transmits neither the modulating signal nor the alarm signal. Therefore, the waveform of the sound is output without being modulated (step S9), and the process is finished. Meanwhile, when the alcohol mode switch is on (YES), the process shifts to step S4.

In step S4, the alcohol sensor 12 detects the alcohol concentration in the exhaled breath (A) and transmits it to the alcohol concentration assessment unit 21 (step S4).

Then, the alcohol concentration assessment unit 21 assesses whether A is higher than the warning threshold (step S5), and transmits the alarm signal when A is higher than the warning threshold (YES). Thereby, the alarm sound is output, and at the same time, the warning image is displayed (S10), and the process is finished. When A is smaller than or equal to the warning threshold (NO), the process moves to step S6.

In step S6, the alcohol concentration assessment unit 21 assesses whether A is higher than the modulation threshold (step S6) and transmits the silent signal when A is smaller than or equal to the modulation threshold (NO). Thereby, the process is finished without the sound being output (step S11). When A is higher than the modulation threshold (YES), the modulating signal is transmitted.

Then, the waveform data generation unit 25 generates the waveform data of the modulated sound based on the data which was converted by the modulation unit 24 to modulate the waveform of the sound, and transmits it to the amplification unit 26. Thereby, the modulated sound is output (step S7), and the process is finished.

While the electrical flute 1 is played, this series of the process may be repeated in a scheduled period. Thereby, the change of the alcohol concentration in the exhaled breath of the user can be reacted sensitively.

Note that the electronic musical instrument of the present invention may not be only a flute but any musical instrument, although the case when the electronic musical instrument was the electrical flute is described in this embodiment. Above all, the alcohol concentration in the exhaled breath can be detected while inputting the sound if it is the musical instrument using the exhaled breath of the user. For example, the electronic musical instrument may be a harmonica, a sax, a trumpet. In the present invention, the sound input into the input means may also be a voice of the people, and the input means may be a microphone.

The present invention can inform the quantity of drinking to the user through the presence or the absence of the modulation of the sound and the alarm sound by the above-mentioned structure. Accordingly, not only the user but also the audiences understand the alcohol concentration of the user and the degree of drunkenness. The user suitably grasps the quantity of drinking by hearing the sound played by themselve and can adjust it. Moreover, because the electrical flute 1 includes not only the alcohol concentration measurement function but also the function to play, its entertainment property is high and it is easy to use during the banquet. Therefore, the quantity of drinking can be shown without giving the user and the audience any load.

### Second Embodiment

The module for electronic musical instruments and the electronic musical instrument of the present invention may further comprise an air conditioning control means to control an outside air conditioning system based on the alcohol concentration detected by the means for detecting the alcohol concentration. If it is such a structure, the air conditioning system (temperature) can be controlled based on the alcohol concentration of the user. Therefore, even if a function of regulate a temperature of a human body is attenuated by the intake of the alcohol, the air conditioning control can be performed to suit the change of the physical condition with the alcohol intake.

As one embodiment of the electronic musical instrument comprising the air conditioning control means, the electrical flute 60 is described below.

Structure of the Electrical Flute 60

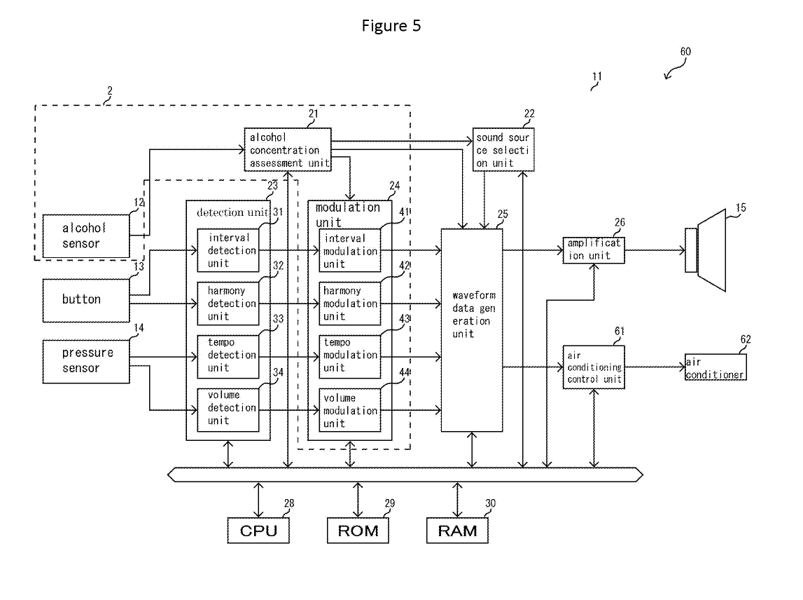

FIG. 5 is a block diagram of the electrical flute 60 which is another embodiment of the electronic musical instrument of the present invention.

It is only different from the electrical flute 1 of the first embodiment that the electrical flute 60 of the present embodiment comprises the air conditioning control unit 61 instead of the image generation unit 27. Therefore, the difference from the first exemplary embodiment is described mainly here and the same number is referred to the component having the function same as the component in the first embodiment to omit the explanation.

The air conditioning control unit 61 is an air conditioning control means to control an outside air conditioning system. The air conditioning system is a system regulating the temperature of the room, and can be an air conditioner such as an air-conditioning apparatuses which are equipped, for example in the room. FIG. 5 shows the case when an outside air conditioner 62 is controlled.

In this embodiment, the Waveform data generation unit 25 transmits an air conditioning control signal to control the air conditioner 62 to the air conditioning control unit 61 based on the signal from the alcohol concentration assessment unit 21. When the silent signal, the modulating signal or the alarm signal described above is received, the waveform data generation unit 25 may transmit the air conditioning control signal and, separately from these, may transmit the air conditioning control signal when the signal indicating the alcohol concentration of the user is received.

For example, the air conditioning control signal may be a signal to make the air conditioner 62 switched on or off, to change a preset temperature to a predetermined value or to turn the preset temperature up or down only the predetermined value. For example, when the alcohol concentration is high, it may be controlled to turn the preset temperature of the air conditioner 62 down only a predetermined value. The preset temperature may also be controlled depending on the alcohol concentration progressively.

The air conditioning control unit 61 controls the air conditioner 62 based on the air conditioning control signal. For example, the air conditioning control unit 61 may transmit the signal to make setting changed to the air conditioner 62 by cable or radio to remote-control the air conditioner 62.

Specific Example of the Flow of the Process

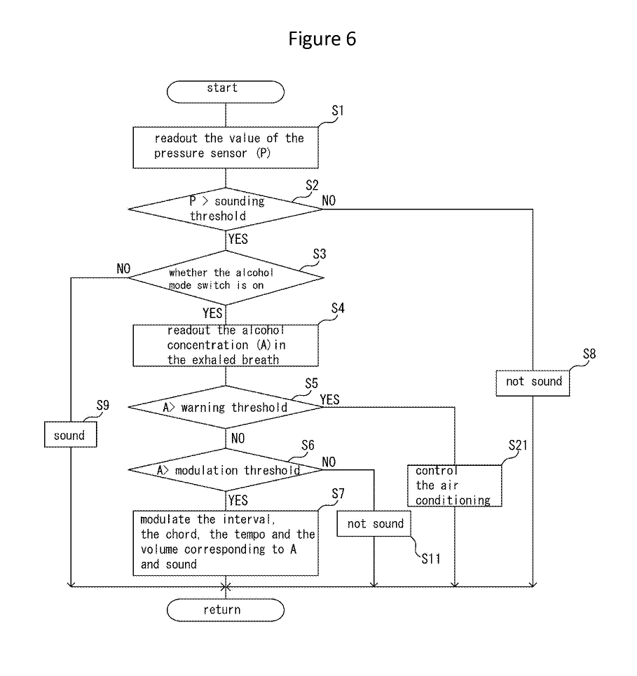

FIG. 6 is a flow chart which shows the example of the flow of the process when the electrical flute 60 of the present exemplary embodiment is used. Note that the explanation is omitted here because step S1-S9 and S11 are the same as step S1-S9 and S11 of the flow of process described in the first exemplary embodiment.

In this embodiment, the alcohol concentration assessment unit 21 transmits the alarm signal to the waveform data generation unit 25, when assessed that the alcohol concentration (A) is higher than the warning threshold in step S5 (YES in step S5). The waveform data generation unit 25 transmits the air conditioning control signal to the air conditioning control unit 61 based on the alarm signal. Thereby, the air conditioner 62 is controlled by the air conditioning control unit 61 (step S21), and the process is finished.

### Third Embodiment

The module for electronic musical instruments and the electronic musical instrument of the present invention may further comprise a light emitting means included in the electronic musical instrument or an outside light emitting means or a light control means to control both lights thereof based on an alcohol concentration detected by the means for detecting the alcohol concentration. If it is such a structure, brightness and a color of the illumination (light) such as the LED which is the light emitting means can be controlled based on an alcohol concentration of a user. Therefore, when the alcohol intakes increase, nuisance is inhibited and their own alcohol intake can be checked visually.

As one embodiment of the electronic musical instrument comprising the light control means, the electrical flute 70 is described below.

Structure of the Electrical Flute 70

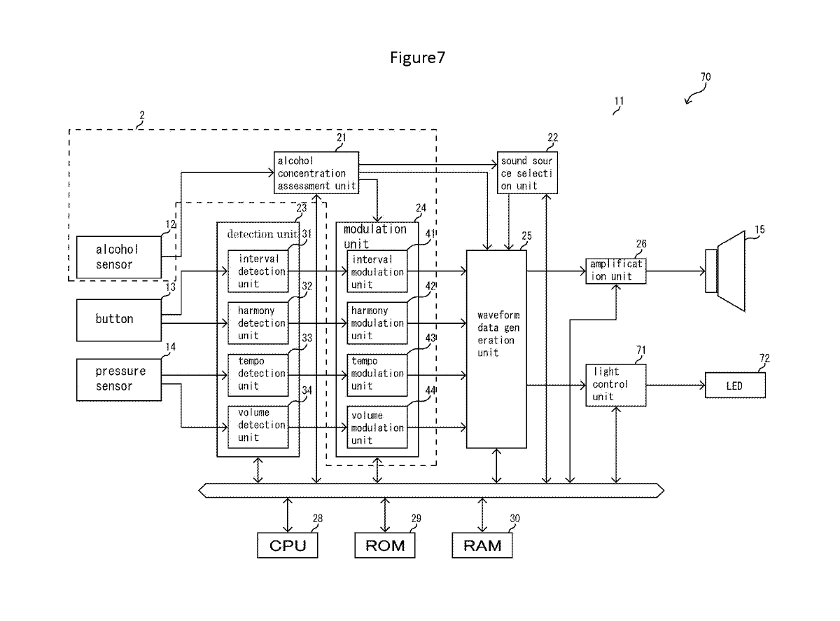

FIG. 7 is a block diagram of the electrical flute 70 which is another embodiment of the electronic musical instrument of the present invention.

It is only different from the electrical flute 1 of the first embodiment that the electrical flute 70 of the present embodiment comprises a light control unit 71 instead of the image generation unit 27. Therefore, the difference from the first embodiment is described mainly here and the same number is referred to the component having the function same as the component in the first exemplary embodiment to omit the explanation.

The light control unit 71 is an electrical light emitting means included in the flute 70 or an outside light emitting means or a light control means to control a presence or an absence of a lighting, a brightness and a color of both lights. For example, the light emitting means can be an electrical warning light which is not shown or a lighting equipment of a room included in the flute 1. FIG. 7 shows in the case when the emission of light of the outside LED72 is controlled.

In this embodiment, the waveform data generation unit 25 transmits the light control signal to control the light to the light control unit 71 based on the signal from the alcohol concentration assessment unit 21. The waveform data generation unit 25 may transmit the light control signal when the silent signal, the modulating signal or the alarm signal described above is received, and separately from these, may transmit the light control signal when the signal indicating the alcohol concentration of the user is received.

For example, the light control signal may be the signal to turn on the LED72, change its brightness, and make it flash or change its color. For example, when the alcohol concentration is high, the blue color light may be turned on in the restraint to inhibit the actions such as nuisance. The brightness and the color of the LED72 may also be controlled depending on the alcohol concentration progressively. The light control signal may be the signal controlling one LED72 and may be the signal controlling a plurality of LED72.

The light control unit 71 controls the emission of the light of the LED72 based on the light control signal. For example, the light control unit 71 may transmit the control signal to the LED72 by cable or radio.

Specific Example of the Flow of the Process

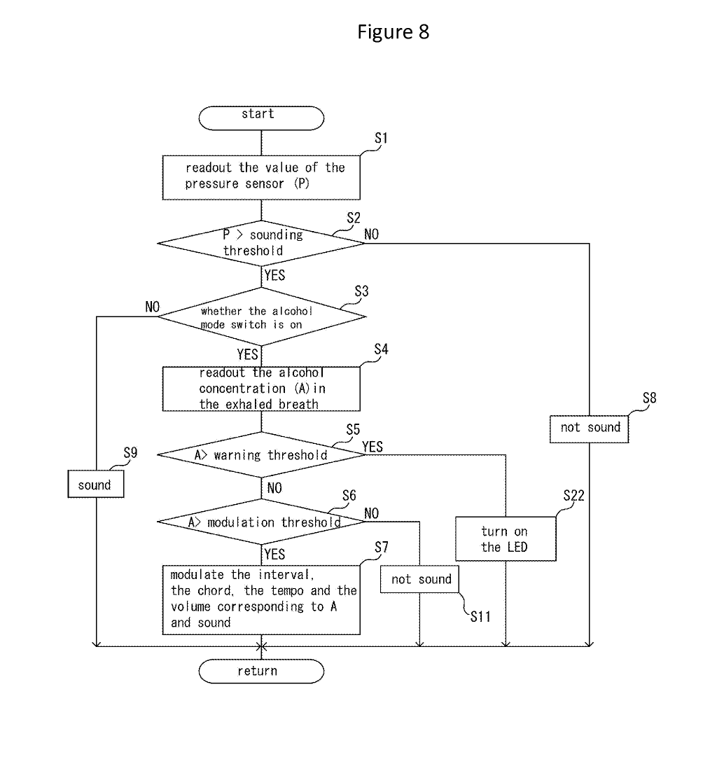

FIG. 8 is a flow chart which shows the example of the flow of the process when the electrical flute 70 of the present exemplary embodiment is used. Note that, because step S1-S9 and S11 are the same as the step S1-S9 and S11 of the flow of process described in first exemplary embodiment, explanation is omitted here.

In this exemplary embodiment, when the alcohol concentration (A) is judged to be higher than the warning threshold in step S5 (YES in step S5), the alcohol concentration assessment unit 21 transmits the alarm signal to the waveform data generation unit 25. The waveform data generation unit 25 transmits the light control signal to the light control unit 71 based on the alarm signal. Thereby, the LED72 is controlled by the light control unit 71 (step S22), and the process is finished.

The present invention is not limited to the embodiment as described above, and various kinds of changes are possible within the scope shown in the claim.

### INDUSTRIAL APPLICABILITY

The present invention can preferably be used for electronic musical instruments and toys, etc.

### DENOTATION OF REFERENCE NUMERALS

1 . . . electrical flute (electronic musical instrument)

2 . . . electrical flute module (module for electronic musical instrument)

11 . . . control circuit board

12 . . . alcohol sensor (means for detecting an alcohol concentration)

13 . . . button (input means)

14 . . . pressure sensor (input means)

15 . . . speaker (output means)

16 . . . display unit

21 . . . alcohol concentration assessment unit

22 . . . sound source selection unit

23 . . . detection unit

24 . . . modulation unit (modulating means)

25 . . . waveform data generation unit

26 . . . amplification unit

27 . . . image generation unit

28 . . . CPU

29 . . . ROM

30 . . . RAM

31 . . . interval detection unit

32 . . . harmony detection unit

33 . . . tempo detection unit

34 . . . volume detection unit

41 . . . interval modulation unit

42 . . . harmony modulation unit

43 . . . tempo modulation unit

44 . . . volume modulation unit

51 . . . warning light

60 . . . electrical flute (electronic musical instrument)

61 . . . air conditioning control unit (air conditioning control means)

62 . . . air conditioner (air conditioning system)

70 . . . electrical flute (electronic musical instrument)

71 . . . light control unit (light control means)

72 . . . LED (light emitting means)

1. A module for electronic musical instruments comprising
an alcohol concentration detection means for detecting an alcohol concentration in an exhaled breath of a user, and
a modulation means for modulating a waveform of a sound based on the alcohol concentration detected by the means for detecting the alcohol concentration.

2. An electronic musical instrument comprising
an input means for inputting the sound to be outputted,
an alcohol concentration detection means for detecting an alcohol concentration in an exhaled breath of a user, and
a modulation means for modulating a waveform of a sound based on the alcohol concentration detected by the means for detecting the alcohol concentration.

3. The module according to claim 1, wherein the modulation means modulates the waveform of the sound by changing at least one or more of an interval, a chord, a tempo, a volume and a type of a musical instrument.

4. The module according to claim 1, further comprising means for controlling an external air conditioning system based on the alcohol concentration detected by the means for detecting the alcohol concentration.

5. The electronic musical instruments according to claim 2, further comprising an air conditioning control means to control an external air conditioning system based on the alcohol concentration detected by the means for detecting the alcohol concentration.

6. The module according to claim 1, further comprising light emitting means included in the electronic musical instrument or external light emitting means.

7. The electronic musical instruments according to claim 2, further comprising light emitting means included in the electronic musical instrument or external light emitting means.

8. The module according to claim 6, further comprising a control means for controlling the light.

9. The electronic musical instruments according to claim 7, further comprising a control means for controlling the light.

10. The electronic musical instrument according to claim 2, wherein the modulating means modulates the waveform of the sound by changing one or more of an interval, a chord, a tempo, a volume and a type of musical instrument.
